(19) United States
(12) Patent Application Publication
(10) Pub. No.: US 2007/0016125 A1
(43) Pub. Date: Jan. 18, 2007

(54) Painless electroporating apparatus

(75) Inventors: Tak-Wah Wong, Kaohsiung City (TW); Sek-Wen Hui, Williamsville, NY (US); Ching-Hung Chen, Yung-Kang City (TW); Chien-Chun Huang, Tainan City (TW); Cheng-De Lin, Chang-Hua Hsien (TW)

Correspondence Address: Paul D. Greeley, Esq.;Ohlandt, Greeley, Ruggiero & Perle, L.L.P., One Landmark Square, 10th Floor, Stamford, CT, 06901-2682, US

(73) Assignee: National Cheng Kung University

(21) Appl. No.: 11/438,893

(22) Filed: May 23, 2006

Related U.S. Application Data
(63) Continuation-in-part of application No. 11/169,874, filed on Jun. 29, 2005, now abandoned.

(51) Int. Cl. A61N 1/30

(52) U.S. Cl. 604/20

(57) ABSTRACT

An electroporating apparatus includes an electroporation electrode device that has a substrate, and an electrode unit provided on the substrate. The electrode unit includes a plurality of positive and negative electrode pads each of which has a skin contact surface area with a width ranging from 0.2 mm to 0.8 mm. Each positive electrode pad is spaced apart from the adjacent negative electrode pad by an electrode spacing of ranging from 0.5 mm to 1.5 mm. The positive and negative electrode pads are needleless. With a decrease in electrode spacing and electrode width, painless electroporation can be achieved when electric pulses of 0.2 ms or less and 150 V or less are applied is to a skin at 10 Hz or less.

### CROSS-REFERENCE TO RELATED APPLICATION

This application is a C-I-P application of U.S. patent application Ser. No. 11/169,874, filed on Jun. 29, 2005.

### BACKGROUND OF THE INVENTION

1. Field of the Invention

The invention relates to an electroporating apparatus for forming electropores in a skin so as to enhance transdermal delivery of drug, more particularly to a painless electroporating apparatus that is used to form electropores in a skin in a painless manner.

2. Description of the Related Art

Administration of therapeutic drugs to patients generally includes oral administration, injection, and transdermal/transmucosal administration. However, oral drugs may cause stomach irritation, whereas injection is painful to the patient. Therefore, the medical field has been endeavoring to develop methods of administering drugs to patients through skin. However, since skin is the most important barrier against bacteria and viruses from invading the human body, therapeutic drugs in general cannot be easily absorbed. In particular, stratum corneum of the skin is a main barrier against absorption of drugs. Therefore, some scholars have developed a method called iontophoresis in which the drug is applied onto the surface of the skin, and an electrode device is used to apply an electric current to the skin such that the drug enters into the human body through electrophoresis and/or electro-osmosis. However, this method is disadvantageous in that the intact skin barrier will obstruct the transport of large quantity or large size drug molecules into the human body so that the therapeutic effect is usually not satisfactory. Besides, the drug may be changed chemically due to electrolysis.

Another method is electroporation, in which perforations are formed in the epidermal, dermal and subcutaneous cells using penetrative needles of an invasive needle electrode assembly, and the drug is introduced through the needles and delivered into the cells and intercellular spaces through the electropores created subsequently in tissue cells by applying pulses across the needle electrode assembly. However, the high voltage electric pulses are transmitted directly to nerve cells of the skin and the muscle, which can induce pain and muscle contraction. An example of such a needle electrode assembly is disclosed in U.S. Pat. No. 6,603,998 of King, in which needle electrodes, coated with solid phase macromolecules, such as DNA, are used for electroporation by penetrating the needle electrodes into an epidermis of a patient. This patent describes that a separation distance between needle electrodes may be in a range of from 50 to 500 microns (0.05 mm to 0.5 mm). Needle electrodes exemplified in this patent have a height of 0.13 mm, a base diameter of 0.043 mm and a tip of less than 0.001 square mm.

Aside from needle electrodes, the prior art has also suggested use of needle-free electrodes for electroporation of a skin. Examples of needle-free electrodes are disclosed in U.S. Pat. Nos. 5,019,034 and 6,748,266. While such needle-free electrodes do not penetrate a skin during use, due to their large electrode sizes and large electrode spacings, the needle-free electrodes still can produce a significant level of pain sensation that makes a patient uncomfortable.

The prior art never suggests that an electrode spacing between needle-free electrodes be reduced to minimize pain sensation. In addition, while the needle electrodes described in U.S. Pat. No. 6,603,998 are sized with small dimensions and small electrode spacing, in view of the penetrative nature of the needle electrodes, use of the small dimensions and electrode spacing of the needle electrodes in this patent is not contemplated to reduce pain. Therefore, nothing disclosed in U.S. Pat. No. 6,603,998 suggests that a decrease in electrode spacing would help the reduction of pain in electroporation.

### SUMMARY OF THE INVENTION

An object of the present invention is to provide an electroporating apparatus with an electroporation electrode device having a reduced electrode spacing between needle-free electrodes and a reduced skin contact surface area so that pain sensation can be minimized or eliminated while still maintaining an enhanced drug delivery efficacy.

According to one aspect of the present invention, an electroporation electrode device comprises: a substrate having top and bottom sides; and an electrode unit provided on said top side of said substrate and including a plurality of positive and negative electrode pads which are adapted to contact a skin and which are arranged in rows, each of said positive and negative electrode pads having a skin contact surface area with a width ranging from 0.2 mm to 0.8 mm, each of said positive electrode pads being spaced apart from an adjacent one of said negative electrodes by an electrode spacing ranging from 0.5 mm to 1.5 mm.

According to another aspect of the present invention, an electroporating apparatus comprises an electrode device that includes a substrate having top and bottom sides, and an electrode unit provided on the top side of the substrate and including a plurality of positive and negative electrode pads which are adapted to contact a skin and which are arranged in rows. Each of the positive and negative electrode pads having a skin contact surface area with a width ranging from 0.2 mm to 0.8 mm. Each of the positive electrode pads being spaced apart from an adjacent one of the negative electrodes by an electrode spacing ranging from 0.5 mm to 1.5 mm. The electroporating apparatus further comprises a pulse generator connected to the electrode device and configured to generate a sequence of electrical pulses adapted to produce a painless electroporation in a skin when the electrode device is placed on the skin.

### BRIEF DESCRIPTION OF THE DRAWINGS

Other features and advantages of the present invention will become apparent in the following detailed description of the preferred embodiments with reference to the accompanying drawings, of which:

FIG. 1 is a perspective view of an electroporation electrode device used in a method embodying the present invention;

FIG. 2 is a top view of the electroporation electrode device of FIG. 1;

FIG. 3 is a fragmentary sectional view taken along line III-III of FIG. 2;

FIG. 4 is a fragmentary sectional view taken along line IV-IV of FIG. 2;

FIG. 5 is a diagram showing the results of tests using different electrode sizes of the electroporation electrode device when pulsing human skin with 60 pulses at 150V, 0.2 ms and a pulse interval of 0.1 and 1.0 s;

FIG. 6 is a diagram showing the results of tests using different electrode spacings of the electroporation electrode device when pulsing with the same conditions as in FIG. 5 employing a pairs of 0.5 mm diameter electrodes; and

FIGS. 7-11 are schematic views showing a method of fabricating the electroporation electrode device.

### DETAILED DESCRIPTION OF THE PREFERRED EMBODIMENTS

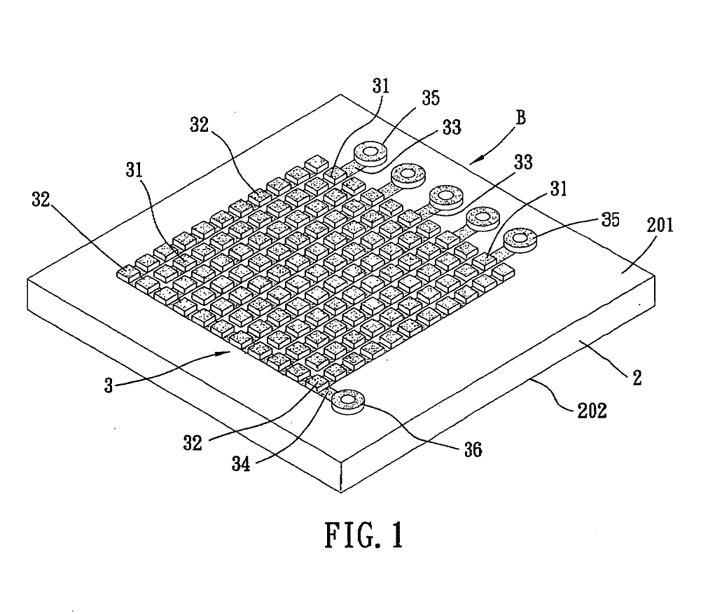

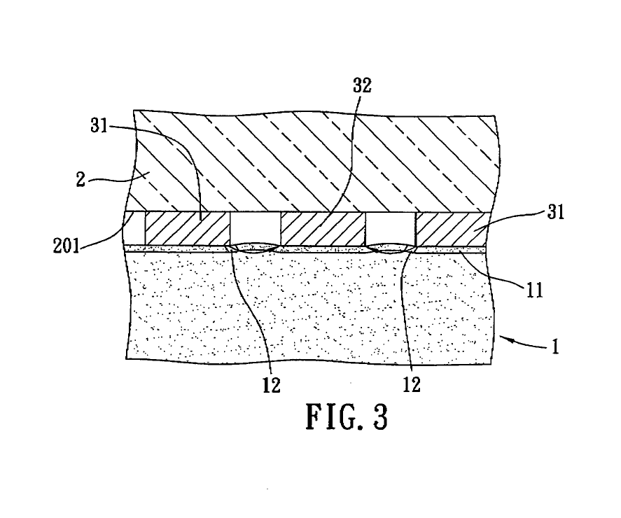

Referring to FIGS. 1 to 4, an electroporating apparatus embodying the present invention is shown to include a pulse generator (A) and an electroporation electrode device (B). The electroporation electrode device (B) includes a substrate 2 formed from an electrical insulating material, and an electrode unit 3 provided on the substrate 2.

The electrode unit 3 includes rows of positive electrode pads 31 and rows of negative electrode pads 32 that are arranged in an alternating manner on a top side 201 of the substrate 2, a plurality of positive connecting plates 33 formed on the top side 201 of the substrate 2 and each electrically connected to all the positive electrode pads 31 in one row, one negative connecting plate 34 formed on the top side 201 of the substrate 2 and connected electrically to all rows of the negative electrode pads 32, a plurality of positive conducting members 35 each extending through the top side 201 and a bottom side 202 of the substrate 2 and connected electrically and respectively to the positive electrode pads 31 in each row through the corresponding positive connecting plate 33, and one negative conducting member 36 which extends through the top side 201 and the bottom side 202 of the substrate 2, which is connected electrically and respectively to all rows of the negative electrode pads 32 through the negative connecting plate 34, and which projects outwardly of the bottom side 202 of the substrate 2.

In a preferred embodiment, the positive and negative electrode pads 31 and 32 are arranged in an 11×11 square matrix. The positive electrode pads 31 are arranged in five rows, and ten positive electrode pads 31 are provided in each row. Five positive conducting members 35 are disposed respectively at front ends of the five rows of positive electrode pads 31. The positive electrode pads 31 in each row are connected to one of the positive conducting members 35 at the front end thereof by a respective one of the positive connecting plates 33 so as to be interconnected electrically.

The negative electrode pads 32 are arranged in six rows. There are eleven negative electrode pads 32 in each row. The six rows of negative electrode pads 32 are arranged alternately with the five rows of positive electrode pads 31. The six rows of negative electrode pads 32 are interconnected electrically through the negative connecting plate 34, and are connected to the negative conducting member 36, which is disposed at a rear edge of the substrate 2.

Five positive connecting plates 33 are used to connect the five rows of positive electrode pads 31 to the respective positive conducting members 35. However, it should be noted that all the positive electrode pads 31 can be connected to only one positive conducting member 35 by a single positive connecting plate 33. Therefore, the number and arrangement of the positive and negative electrode pads 31, 32 can be varied depending on drug administration requirements, and should not be limited to the foregoing. In addition, the manner of connection of the positive and negative connecting plates 33, 34 can be adjusted to enable electrical connection of some or all of the positive and negative electrode pads 31, 32.

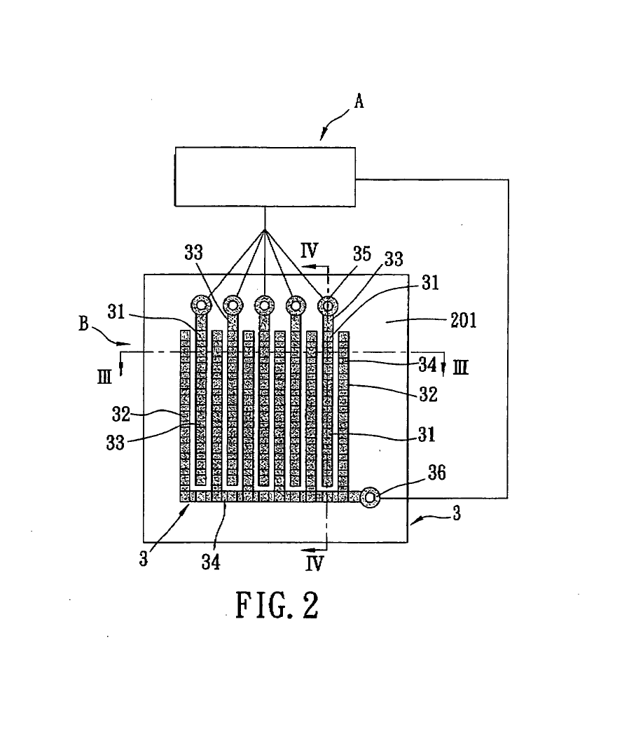

Referring once again to FIG. 2, the pulse generator (A) is connected electrically to the positive and negative conducting members 35 and 36. The pulse generator (A) is configured to generate a sequence of electrical pulses adapted to produce a painless electroporation in a skin when the electrode device (B) is placed on the skin. Preferably, the electric pulses have a pulse width not larger than 0.2 ms and a peak voltage not larger than 150V and are produced at a frequency of 1 to 10 Hz.

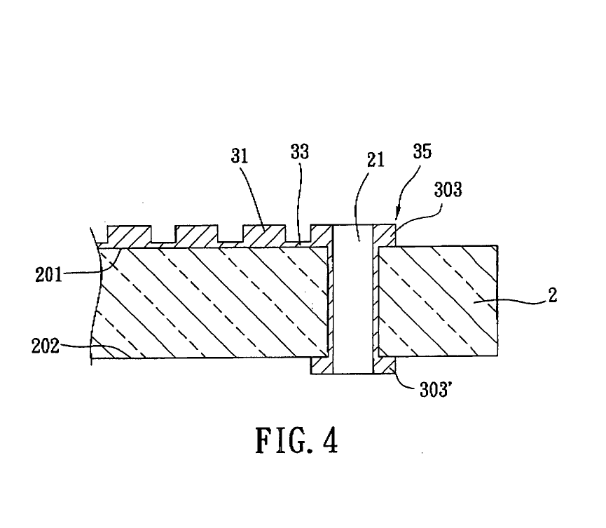

Referring to FIG. 4 in combination with FIG. 2, the positive and negative conducting members 35, 36 are hollow tubular structures. The electric pulse signal is conducted from the bottom side 202 of the substrate 2 to the top side 201 of the substrate 2 for transmission to the positive and negative electrode pads 31, 32 through the positive and negative conducting plates 33, 34. FIG. 4 illustrates the structures and connective relationship of the positive conducting member 35, the positive connecting plate 33, and the positive electrode pads 31. The structures and connective relationship of the negative conducting member 36, the negative connecting plate 34, and the negative electrode pads 32 are not illustrated therein as they are substantially similar to those of the positive conducting member 35, the positive connecting plate 33, and the positive electrode pads 31.

According to the present invention, each of the positive and negative electrode pads 31, 32 is provided with a small skin contact surface area and a small electrode spacing between each positive electrode pad 31 and an adjacent negative electrode pad 32. In particular, the width of the skin contact surface area is set to be in a range of from 0.2 mm to 0.8 mm, preferably 0.3 mm to 0.6 mm. If the width is smaller than 0.2 mm, the positive and negative electrode pads 31, 32 will penetrate a skin and produce a prickly sensation like needle electrodes. If the width is larger than 0.8 mm, pain sensation cannot be reduced. The electrode spacing is arranged to be in a range of from 0.5 mm to 1.5 mm, preferably 0.6 mm to 1 mm. If the electrode spacing is smaller than 0.5 mm, short circuits are likely to occur during application of electrical pulses. If the electrode spacing is larger than 1.5 mm, unacceptable pain can result. In a preferred embodiment, the positive and negative electrode pads 31, 32 are configured to be square in shape for purposes of simplifying fabrication. The width of the square skin contact surface area ranges from 0.5 to 0.6 mm, the electrode spacing is 0.6 mm, and the height/thickness of the positive and negative electrode pads 31, 32 is about 0.2 mm. However, it is contemplated that the positive and negative electrode pads 31, 32 could be provided with any other suitable shapes.

In use, the top side 201 of the substrate 2 is pressed against the surface of the skin 1. Electrical pulses are applied to the skin 1 through the positive electrode pads 31. The electric pulse signals may be square wave pulses, exponential decay pulses, or AC pulses. The voltage may be at least more than 50V. Each pulse may be maintained for a duration of 0.2 ms or less, and the interval between pulses may be 0.1 second or longer.

The electroporation electrode device of the present invention may be used for administrating various therapeutic agents, such as anesthetics, antibiotics, hormones, chemotherapy agents, nucleic acid sequences, peptides, protein, various vaccine or serum combinations, etc.

Tests of Pain Levels Using Electrode Pads with Different Sizes and Different Electrode Spacings

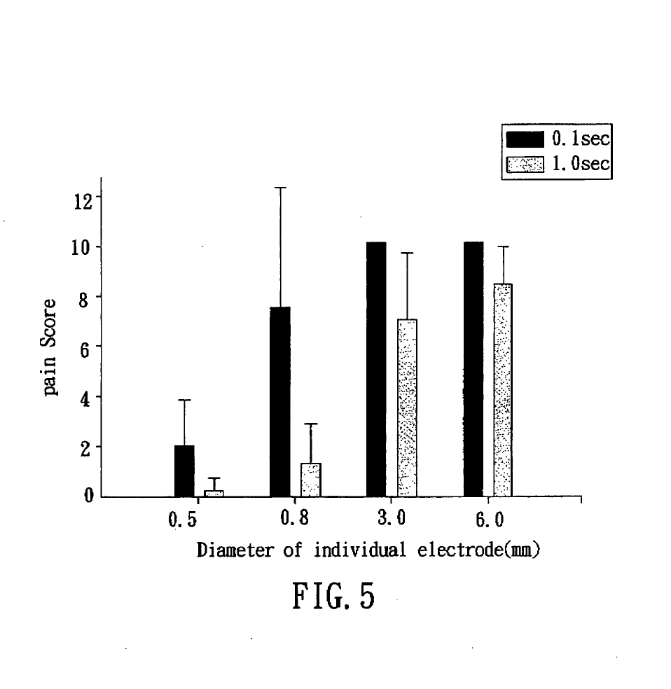

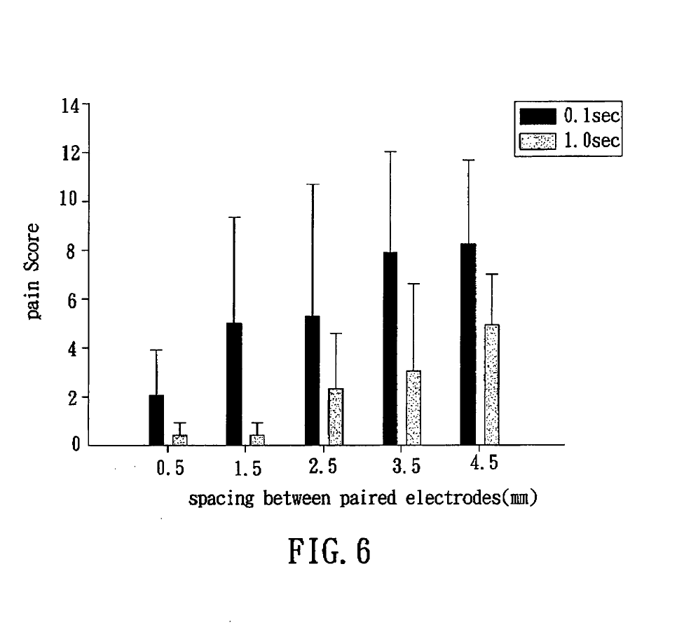

Electrodes pads having different sizes and different electrode spacings were tested on five healthy young adult volunteers' forearm with non-sun exposed skin. A pulse generator was used to produce multiple unipolar square pulses up to 150V at different frequencies and pulse widths for application of electroporation pluses. The results are shown in FIGS. 5 and 6. Pain scores higher than 5 were defined as unacceptable.

To examine the effects of the different sizes of the electrode pads, pairs of stainless steel cylinders having different diameters were used as electrode pads. Sixty electric pulses having 150V, a pulse width 0.2 ms, and a pulse interval of 0.1 second and 1 second were applied to the skins of forearms of the young adult volunteers. FIG. 5 shows that, when all conditions are equal, pulsing with a larger electrode on the skins tends to induce more pain. FIG. 5 further shows that, when the electrode width or diameter is larger than 0.8 mm, the pain score is unacceptable.

To investigate the effects of the different electrode spacings, pairs of cylindrical electrode pads of 0.5 mm diameter separated at different electrode spacings were used. FIG. 6 shows that, for the same electrode size (diameter), the pain score decreases when the electrode spacing is reduced. This is because electrode pads of opposite polarity having shorter electrode spacings produce shallower electric fields, thus stimulating only shallower layers of a skin containing pain-sensing nerve endings. Use of a smaller electrode spacing can eliminate muscle twitch that was produced by deeply penetrating electric pulses when a longer electrode spacing is used. With electrode pads having an electrode spacing of 0.5 mm, the pain score is below the threshold of sensation. However, when the electrode spacing is less than 0.5 mm, short circuits are prone to occur during pulsing on some moist skin. FIG. 6 further shows that, when the electrode spacing is larger than 1.5 mm, unacceptable pain is produced.

The results of the aforesaid tests further show that, when both of the electrode spacing and the width of the electrode pads reach about 0.5 mm, the pain level is not perceptible even at the threshold of transdermal electroporation level of sixty electric pulses having 150V and 0.2 ms at 1-10 Hz.

A method for fabricating the electroporation electrode device of the present invention is described as follows:

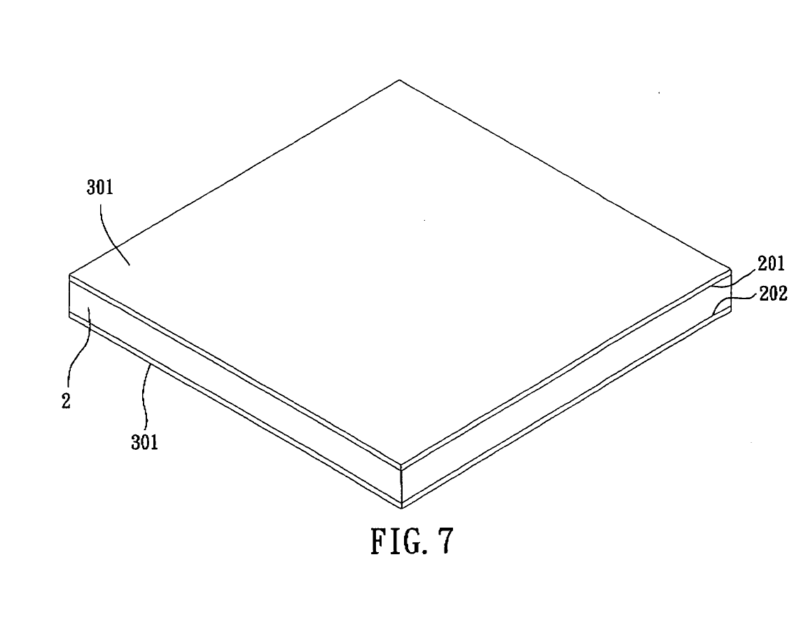

Referring to FIG. 7, a thick metal plate layer 301 having a thickness of 0.2 mm is plated on each of the top side 201 and the bottom side 202 of the insulating substrate 2. In this embodiment, the thick metal plate layer 301 is formed from copper.

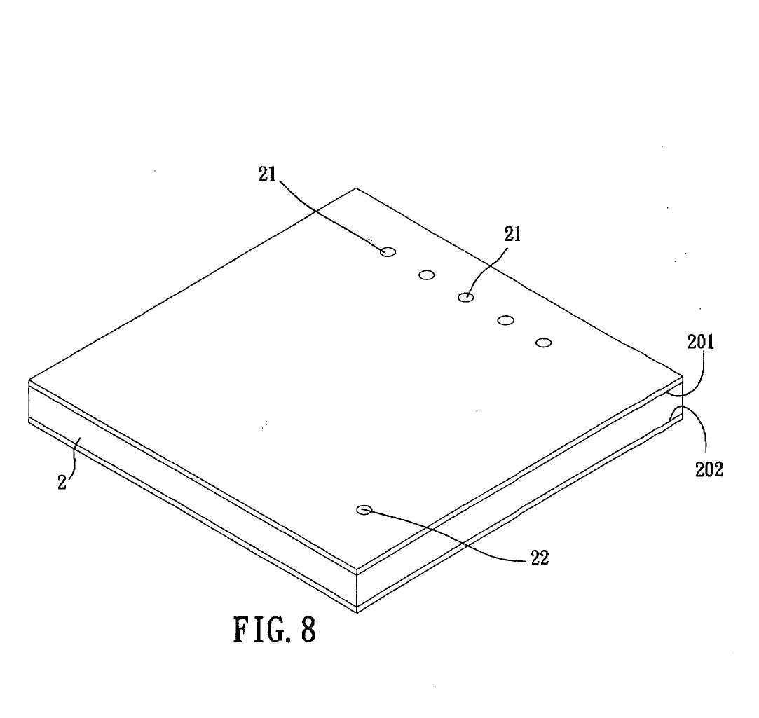

Referring to FIG. 8, a front part of the substrate 2 is drilled to form five transversely spaced-apart through holes 21. A rear part of the substrate 2 is drilled to form a through hole 22.

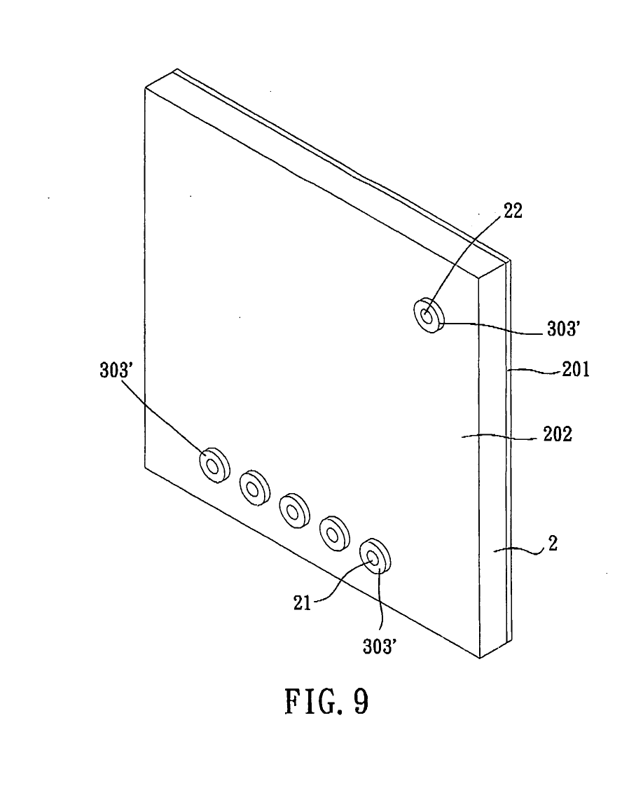

Referring to FIG. 9, the thick metal plate layer 301 on the bottom side 202 of the substrate 2 is removed in part by etching or engraving such that a ring-shaped metal plate 303′ is formed around each of the through holes 21, 22 on the bottom side 202.

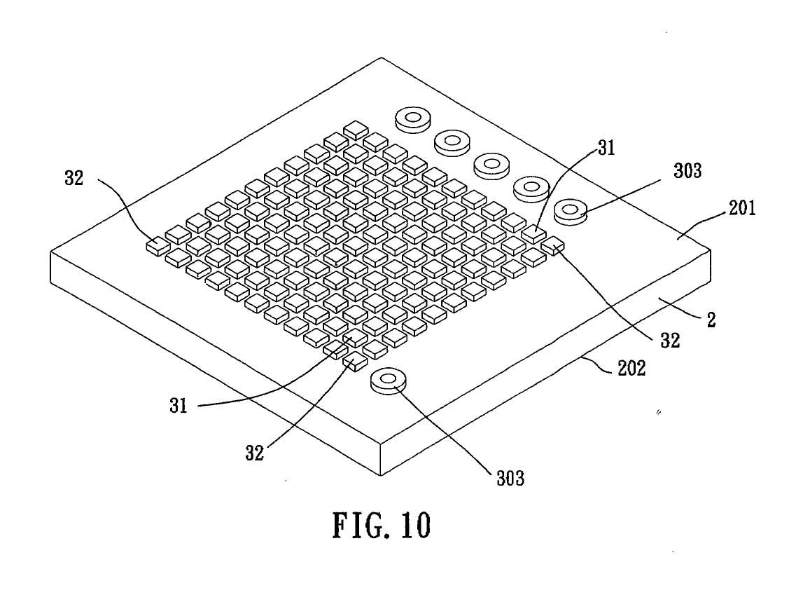

Referring to FIG. 10, the thick metal plate layer 301 on the top side 201 of the substrate 2 is engraved using an engraving machine (not shown) to form the positive electrode pads 31, the negative electrode pads 32, and ring-shaped metal plates 303 around the through holes 21, 22 on the top side 201 such that the positive and negative electrode pads 31, 32 are isolated electrically from each other and are alternately arranged.

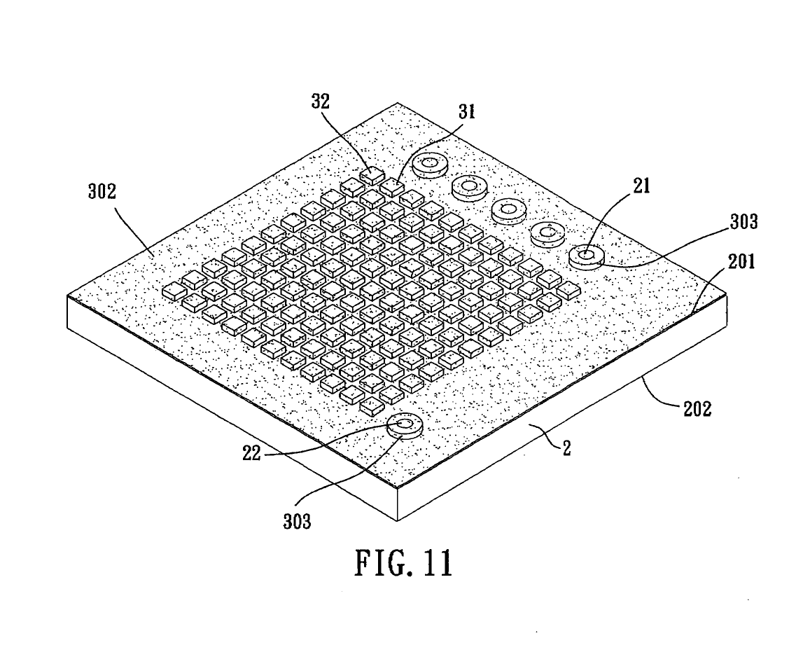

Referring to FIG. 11, a thin metal layer 302 is electroplated on the top side 201 of the substrate 2 using copper sulfate as the electroplating solution such that inner walls defining the through holes 21, 22 are also plated with the thin metal layer 302 to enable electrical connection between the metal plates 303, 303′ on the top and bottom sides 201, 202 of the substrate 2, thereby forming the positive and negative conducting members 35, 36 as shown in FIG. 1.

Thereafter, the thin metal plate layer 302 between each positive electrode pad 31 and the negative electrode pad(s) 32 adjacent thereto is removed by etching such that each row of the positive electrode pads 31 is connected electrically to the respective positive conducting member 35 and such that each row of the negative electrode pads 32 is connected electrically to the negative conducting member 36, with the positive and negative electrode pads 31, 32 isolated electrically from each other.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

We claim:

1. An electroporation electrode device for painlessly electroporating a skin or mucosa so as to enhance transdermal delivery of cosmetic or therapeutic molecules comprising:
a substrate having top and bottom sides; and
an electrode unit provided on said top side of said substrate and including a plurality of positive and negative electrode pads which are adapted to contact a skin and which are arranged in rows, each of said positive and negative electrode pads having a skin contact surface area with a width ranging from 0.2 mm to 0.8 mm, each of said positive electrode pads being spaced apart from an adjacent one of said negative electrodes by an electrode spacing ranging from 0.5 mm to 1.5 mm.

2. The electroporation electrode device as claimed in claim 1, wherein said electrode spacing is 0.6 mm-1 mm.

3. The electroporation electrode device as claimed in claim 2, wherein said width of said skin contact surface area ranges from 0.3 mm to 0.6 mm.

4. The electroporation electrode device as claimed in claim 3, wherein each of said positive and negative electrode pads has a height of about 0.2 mm measured from said top side of said substrate.

5. The electroporation electrode device as claimed in claim 1, wherein the rows of said positive electrode pads are arranged alternately with the rows of said negative electrode pads.

6. The electroporation electrode device as claimed in claim 5, wherein said electrode unit further includes a plurality of positive connecting plates and a plurality of positive conducting members, each of said connecting plates being connected to all of said positive electrode pads in each row and one of said positive conducting members.

7. The electroporation electrode device as claimed in claim 6, wherein said electrode unit further includes one negative connecting plate and one negative conducting member, said negative conducting member being connected to all rows of said negative electrode pads through said negative connecting plate.

8. The electroporation electrode device as claimed in claim 7, wherein each of said negative and positive conducting members includes a conductive through hole extending through said top and bottom sides, a top ring-shaped metal plate formed on said top side and connected electrically to said through hole, and a bottom ring-shaped metal plate formed on said bottom side and connected electrically to said through hole.

9. An electroporating apparatus comprising:
an electrode device that includes a substrate having top and bottom sides, and an electrode unit provided on said top side of said substrate and including a plurality of positive and negative electrode pads which are adapted to contact a skin and which are arranged in rows, each of said positive and negative electrode pads having a skin contact surface area with a width ranging from 0.2 mm to 0.8 mm, each of said positive electrode pads being spaced apart from an adjacent one of said negative electrodes by an electrode spacing ranging from 0.5 mm to 1.5 mm; and a pulse generator connected to said electrode device and configured to generate a sequence of electrical pulses adapted to produce a painless electroporation in a skin when said electrode device is placed on the skin.

10. The electroporating apparatus of claim 9, wherein said electric pulses have a pulse width not larger than 0.2 ms and a peak voltage not larger than 150V and are produced at a frequency not higher than 10 Hz.
